(19) United States
(12) Patent Application Publication
(10) Pub. No.: US 2006/0150987 A1
(43) Pub. Date: Jul. 13, 2006

(54) Operating room draping system and method for using the same

(76) Inventors: Mark S. Dillon, Columbus, MS (US); Denise Oxley, Holts Summil, MO (US); Cindy Henke, Wheat Ridge, CO (US)

Correspondence Address: WITHERS & KEYS, LLC, P. O. BOX 2049, MCDONOUGH, GA, 30253, US

(21) Appl. No.: 11/240,951

(22) Filed: Sep. 30, 2005

Related U.S. Application Data
(60) Provisional application No. 60/615,237, filed on Oct. 1, 2004.

(51) Int. Cl. A61B 19/00; A61B 19/08; A61F 5/37

(52) U.S. Cl. 128/849; 128/853

(57) ABSTRACT

Disposable operating room draping systems are disclosed. Methods of using disposable operating room draping systems are also disclosed.

### FIELD OF THE INVENTION

The present invention relates generally to disposable operating room surgical draping systems, and methods for using disposable operating room surgical draping systems in an operating room setting.

### BACKGROUND OF THE INVENTION

A variety of disposable drapes and linens are used in operating rooms. Disposable drapes and linens may be used to protect and/or cover a patient, to protect and/or cover a piece of operating room equipment, or both. During surgical procedures, it is important to maintain a sterile field in areas of the operating room. The sterile field may be located near or on a patient, a piece of equipment, or both. Efforts continue in the design of draping systems for a draping system that is able to provide (i) superior protection of a patient and/or a piece of equipment, as well as (ii) an acceptable sterile field.

What is needed in the art is a disposable operating room draping system and a method of using such a system so as to (i) provide superior protection of a patient and/or a piece of equipment, (ii) provide an acceptable sterile field near or on a patient and/or a piece of equipment, or (iii) both (i) and (ii).

### SUMMARY OF THE INVENTION

The present invention is directed to a disposable drape suitable for use in an operating room setting. The disposable drape of the present invention addresses one or more of the above-mentioned deficiencies in the art. In particular, the disposable drape of the present invention provides one or more of the following features: (i) superior protection of a patient and/or a piece of equipment, and (ii) the ability to form an acceptable sterile field near or on a patient, near or on a piece of equipment, or through a piece of equipment.

According to one exemplary embodiment of the present invention, the disposable operating room drape comprises a first layer of material having a first outer periphery and a first fenestration having a first fenestration area positioned within the first outer periphery; a second layer of material having a second outer periphery and a second fenestration positioned within the second outer periphery, wherein the second layer of material is joined to the first layer of material along a portion of the second outer periphery; and a collar member having an inner periphery and an outer periphery, wherein the inner periphery of the collar member is attached to the second layer of material so as to surround the second fenestration of the second layer of material; wherein the collar member is releasably attachable to the first layer of material. In this embodiment, the collar member may extend through the first fenestration alone or through both the first and second fenestrations to be releasably attachable to the first layer of material.

According to a further exemplary embodiment of the present invention, the disposable operating room drape comprises a first layer of material having a first outer periphery and a first fenestration having a first fenestration area positioned within the first outer periphery; a second layer of material having a second outer periphery and a second fenestration positioned within the second outer periphery, wherein the second layer of material is joined to the first layer of material along a portion of the second outer periphery; and an adhesive film attached to an upper surface of the first layer of material and extending over a portion of the first fenestration area, wherein the adhesive film is releasably attachable to the second layer of material through the first fenestration.

In a further exemplary embodiment of the present invention, the disposable operating room drape comprises an operating room drape having a cuff at one end and on a lower surface of the operating room drape, wherein the cuff is sized so as to be capable of partially enclosing a portion of an operating room table mattress, and wherein the operating room drape has a first fenestration therein, and the cuff has a second fenestration therein, and the first and second fenestrations are aligned with one another.

The disposable operating room drapes of the present invention may be used alone or in combination with other drapes to create drape systems suitable for use in an operating room setting. One exemplary disposable operating room draping system of the present invention comprises (a) a first disposable drape comprising (i) a first layer of material having a first outer periphery and a first fenestration having a first fenestration area positioned within the first outer periphery, and (ii) a second layer of material having a second outer periphery and a second fenestration positioned within the second outer periphery, wherein the second layer of material is joined to the first layer of material along a portion of the second outer periphery so as to form a cuff, wherein the cuff is sized so as to be capable of partially enclosing a portion of an operating room table mattress; and (b) a second disposable drape comprising (i) a third layer of material having a third outer periphery, and (ii) a fourth layer of material attached to the third layer of material and positioned within the third outer periphery, wherein the fourth layer of material is capable of absorbing liquid. Such draping systems may be used in a variety of surgical procedures to create relatively large sterile fields in an operating room setting.

The present invention is further directed to methods of preparing an operating room substrate for a surgical procedure. In one exemplary embodiment of the present invention, the method of preparing an operating room substrate comprises the steps of: placing a disposable drape of the present invention onto an outer surface of a substrate having a substrate fenestration therein; aligning a first fenestration of the disposable drape with the substrate fenestration of the operating room substrate; and releasably attaching a portion of an adhesive film located on a first layer of material of the disposable drape to a second layer of material of the disposable drape through the first fenestration and the substrate fenestration. In one exemplary embodiment of the present invention, the operating room substrate comprises an operating room table.

The present invention is even further directed to methods of creating a sterile field in an operating room setting. The sterile field may be near or on a patient, near or on a piece of equipment, or through a piece of equipment, such as an operating room table. In one exemplary embodiment of the present invention, the method of creating a sterile field comprises the steps of placing a disposable drape of the present invention onto an outer surface of an operating room substrate having a substrate fenestration therein; aligning a first fenestration of the disposable drape with the substrate fenestration of the operating room substrate; either (i) releasably attaching a portion of a collar member extending from a second layer of material of the disposable drape beneath the operating room substrate to a first layer of material of the disposable drape through the first fenestration and the substrate fenestration, or (ii) releasably attaching a portion of an adhesive film located on a first layer of material of the disposable drape to a second layer of material of the disposable drape through the first fenestration and the substrate fenestration; and extending a portion of a patient's body through the first fenestration and the substrate fenestration. In one exemplary embodiment of the present invention, the operating room substrate comprises an operating room table.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a top view of an exemplary disposable drape of the present invention;

FIG. 2 depicts a rear view of the exemplary disposable drape of FIG. 1;

FIG. 3 depicts a cross-sectional view of the exemplary disposable drape of FIG. 1 along line A-A shown in FIG. 1;

FIG. 4 depicts a cross-sectional view of the exemplary disposable drape of FIG. 1 along line B-B shown in FIG. 1;

FIG. 5 depicts a cross-sectional view of the exemplary disposable drape of FIG. 1 along line A-A shown in FIG. 1 after a portion of an adhesive film of the exemplary disposable drape is releasably attached to a portion of the second layer of material of the exemplary disposable drape;

FIG. 6 depicts a top view of another exemplary disposable drape of the present invention;

FIG. 7 depicts a rear view of the exemplary disposable drape of FIG. 6;

FIG. 8 depicts a cross-sectional view of the exemplary disposable drape of FIG. 6 along line G-G shown in FIG. 6;

FIG. 9 depicts a cross-sectional view of the exemplary disposable drape of FIG. 6 along line G-G shown in FIG. 6 after a portion of a collar member of the exemplary disposable drape is releasably attached to a portion of the first layer of material of the exemplary disposable drape;

FIG. 10 depicts a top view of an exemplary disposable drape suitable for use in an exemplary disposable operating room surgical draping system of the present invention;

FIG. 11 depicts a side view of the exemplary disposable drape of FIG. 10 as viewed from position E shown in FIG. 10;

FIG. 12 depicts a side view of an exemplary piece of operating room equipment that may be at least partially covered with the disposable drapes or drape systems of the present invention;

FIG. 13 depicts a side view of the exemplary piece of operating room equipment shown in FIG. 12 after being at least partially covered with an exemplary disposable drape system of the present invention; and

FIG. 14 depicts a side view of the exemplary piece of operating room equipment shown in FIG. 12 after being at least partially covered with another exemplary disposable drape system of the present invention.

### DETAILED DESCRIPTION OF THE INVENTION

The present invention is directed to disposable operating room drapes and draping systems for use in an operating room setting. The present invention is further directed to methods of using disposable operating room drapes and draping systems to properly prepare an operating room setting prior to exposing the setting to a patient. In addition, the present invention is directed to methods of using disposable operating room drapes and draping systems to prepare sterile fields within an operating room setting.

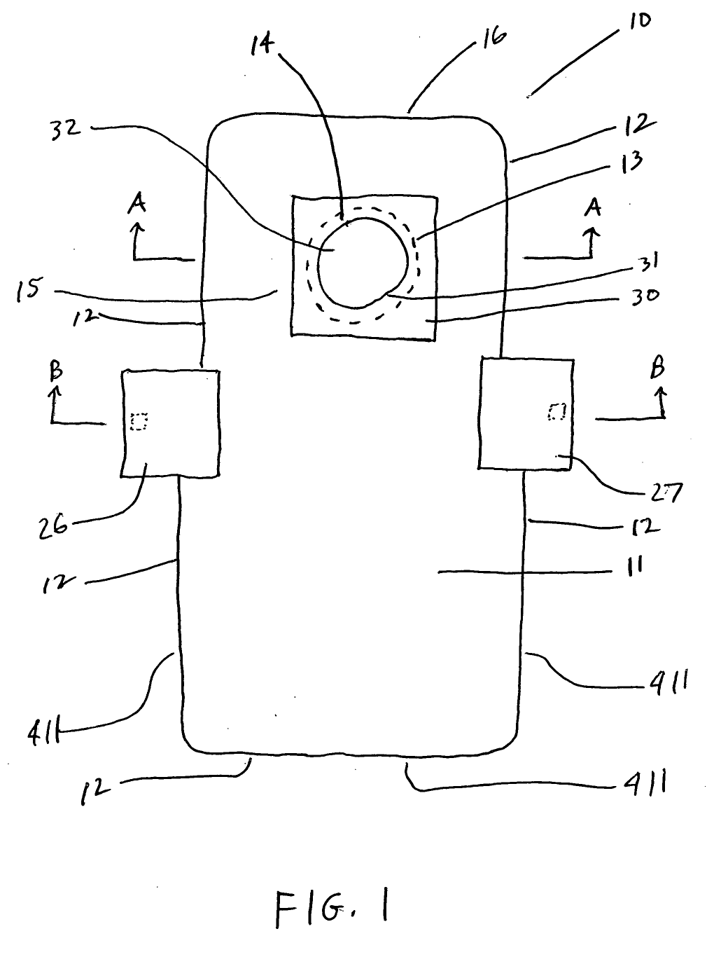

In one embodiment of the present invention, the disposable operating room drape comprises a first layer of material having a first outer periphery and a first fenestration having a first fenestration area positioned within the first outer periphery; a second layer of material having a second outer periphery and a second fenestration positioned within the second outer periphery, wherein the second layer of material is joined to the first layer of material along a portion of the second outer periphery; and an adhesive film attached to an upper surface of the first layer of material and extending over a portion of the first fenestration area, wherein the adhesive film is releasably attachable to the second layer of material through the first fenestration. Such an exemplary disposable operating room drape is shown in FIGS. 1-5.

As shown in FIG. 1, exemplary disposable operating room drape 10 comprises a first layer of material 11 having a first outer periphery 12 and a first fenestration 13 having a first fenestration area 14 positioned within first outer periphery 12. An outer periphery of first fenestration 13 is shown by the dashed circular line in FIG. 1. First fenestration area 14 comprises the area bond by the circular dashed line in FIG. 1. First layer of material 11 may comprise one or more layers bonded to one another, and have a number of additional features as described below including, but not limited to, wings 26 and 27 extending from opposite sides of first layer of material 11.

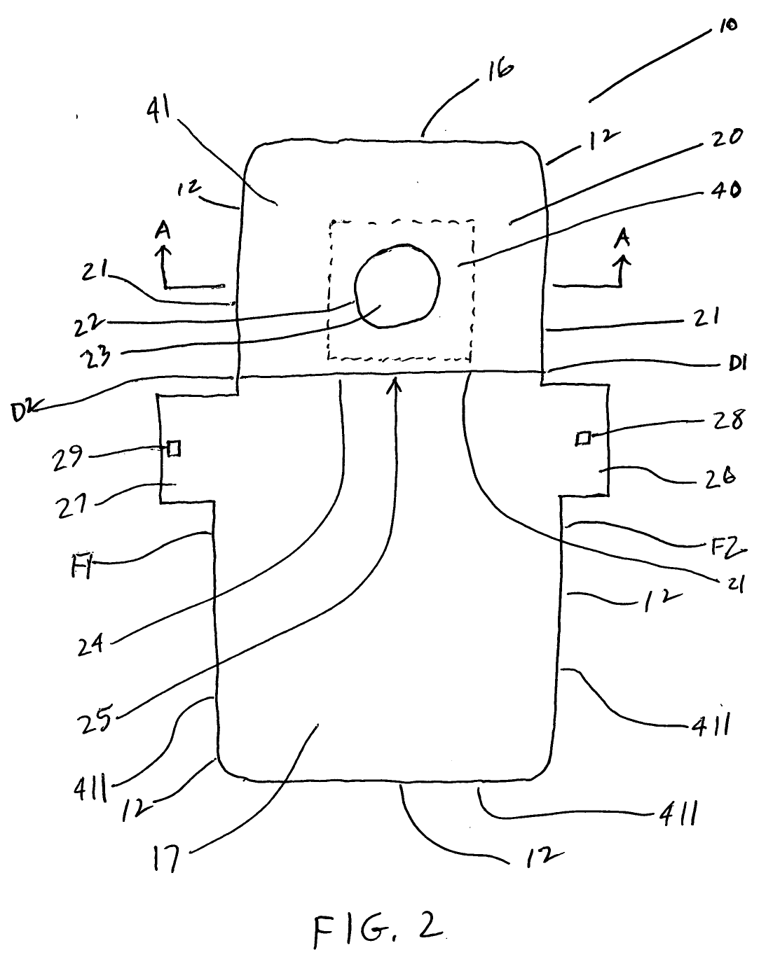

As shown in FIG. 2, exemplary disposable operating room drape 10 comprises a second layer of material 20 having a second outer periphery 21 and a second fenestration 22 having a second fenestration area 23 positioned within second outer periphery 21. An outer periphery of second fenestration 22 is shown by the solid circular line in FIG. 2. Second fenestration area 23 comprises the area bond by the solid circular line in FIG. 2. Second layer of material 20 of exemplary disposable operating room drape 10 is joined to first layer of material 11 along at least a portion of second outer periphery 21 from point D1 to point D2 along second outer periphery 21.

In one desired embodiment of the present invention, second layer of material 20 forms cuff 41, which partially covers a lower surface 17 of first layer of material 11, wherein cuff 41 has a closed end 16 and an open end 24 extending across a width of exemplary disposable operating room drape 10 from point D1 to point D2. Open end 24 of cuff 41 may be separated from lower surface 17 of first layer of material 11 to access pocket 25 between second layer of material 20 and first layer of material 11. Cuff 41 and pocket 25 contained therein are desirably sized so as to fit over at least a portion of an operating room table having an opening therein, a table mattress having an opening therein, or both.

Referring again to FIG. 1, exemplary disposable operating room drape 10 further comprises an adhesive film 30 attached to an upper surface 15 of first layer of material 11. At least a portion of adhesive film 30 extends over a portion of first fenestration area 14. Adhesive film 30 comprises an adhesive film fenestration 31 having adhesive film fenestration area 32. An outer periphery of adhesive film fenestration 31 is shown by the solid circular line in FIG. 1. Adhesive film fenestration area 32 comprises the area bond by the solid circular line in FIG. 1. Adhesive film 30 is releasably attachable to second layer of material 20 through first fenestration 13.

Referring again to FIG. 2, exemplary disposable operating room drape 10 further comprises a release liner 40 applied over and temporarily attached to any portion of adhesive film 30 that extends over first fenestration area 14. Release liner 40 is positioned between lower surface 17 of first layer of material 11 and second layer of material 20 within pocket 25 of cuff 41. An outer periphery of release liner 40 is shown by the dashed square line in FIG. 2. By removing release liner 40, adhesive film 30 is capable of being releasably attached to second layer of material 20 through first fenestration 13.

A cross-sectional view of exemplary disposable drape 10 viewed along line A-A of FIG. 1 is provided in FIG. 3. As shown in FIG. 3, exemplary disposable drape 10 comprises an uppermost adhesive film 30 positioned on upper surface 15 of first layer of material 11. Adhesive film 30 has adhesive film fenestration 31 having a cross-sectional dimension, d₁. First layer of material 11 comprises first fenestration 13 having a cross-sectional dimension, d₂, wherein d₂ has a greater length than d₁. This enables at least a portion of adhesive film 30 to extend over first fenestration area 14.

Release liner 40 is adjacent to lower surface 17 of first layer of material 11, and releasably adhered to portions of adhesive film 30 that extend over first fenestration area 14. Second layer of material 20 comprising second fenestration 22 having a cross-sectional dimension, d₃, is positioned below release liner 40 and lower surface 17 of first layer of material 11. In exemplary disposable drape 10, cross-sectional dimension, d₃, has a length substantially equal to d₁. As shown in FIG. 3, pocket 25 of exemplary disposable drape 10 is between first layer of material 11 and second layer of material 20 from a first side of outer periphery 21 to an opposite second side of outer periphery 21.

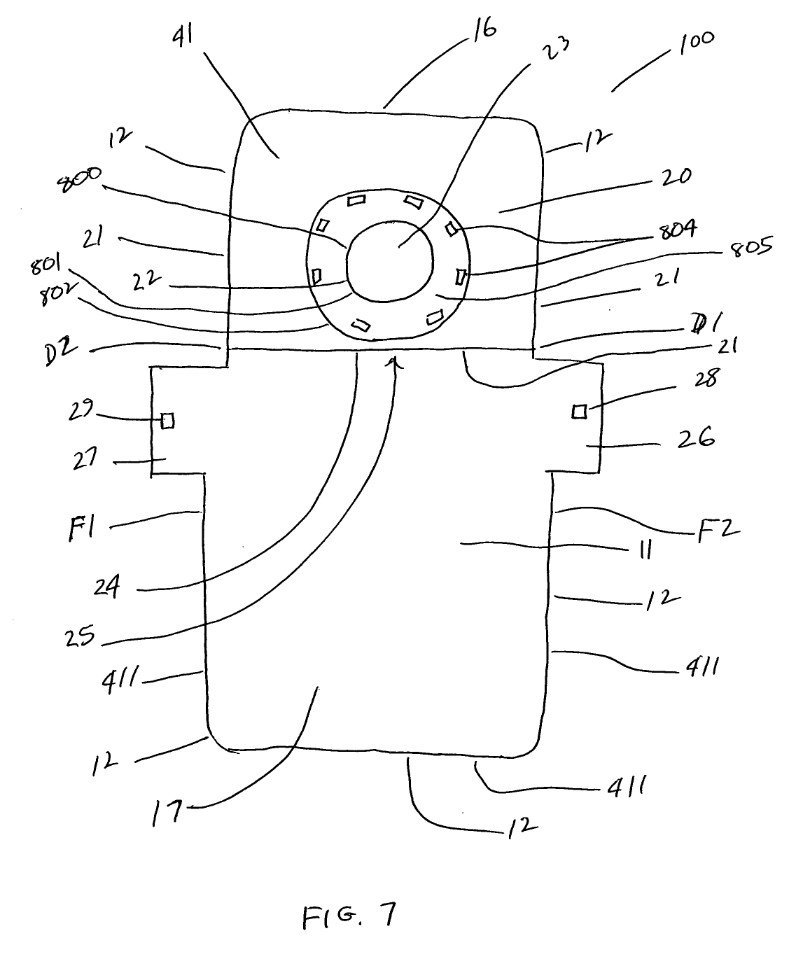

In a further embodiment of the present invention, the disposable operating room drape comprises a first layer of material having a first outer periphery and a first fenestration having a first fenestration area positioned within the first outer periphery; a second layer of material having a second outer periphery and a second fenestration positioned within the second outer periphery, wherein the second layer of material is joined to the first layer of material along a portion of the second outer periphery; and a collar member having a first end and a second end, wherein the first end of the collar member is attached to the second layer of material so as to surround the second fenestration of the second layer of material; wherein the collar member is releasably attachable to the first layer of material. Such an exemplary disposable operating room drape is shown in FIGS. 6-10.

As shown in FIG. 6, exemplary disposable operating room drape 100 comprises a first layer of material 11 having a first outer periphery 12 and a first fenestration 13 having a first fenestration area 14 positioned within first outer periphery 12. First layer of material 11 may comprise one or more layers bonded to one another, and have a number of additional features as described below including, but not limited to, wings 26 and 27 extending from opposite sides of first layer of material 11.

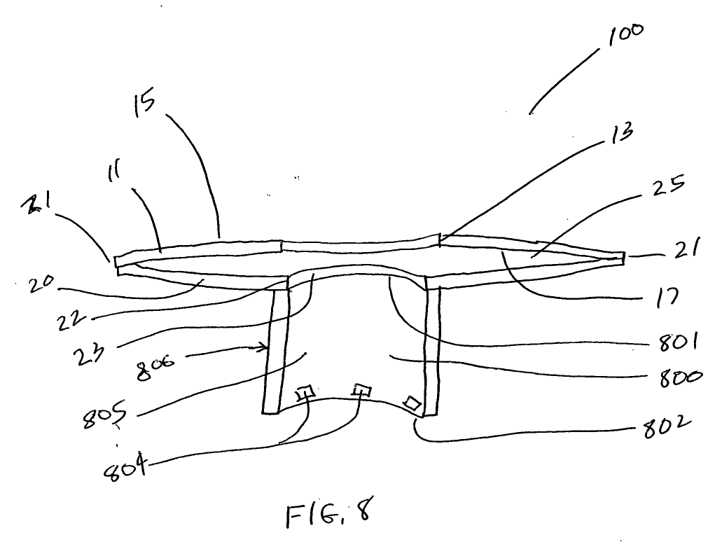

As shown in FIG. 7, exemplary disposable operating room drape 100 comprises a second layer of material 20 having a second outer periphery 21 and a second fenestration 22 having a second fenestration area 23 positioned within second outer periphery 21. An outer periphery of second fenestration 22 is shown by the solid circular line in FIG. 2. Second fenestration area 23 comprises the area enclosed by the solid circular line in FIG. 2. Second layer of material 20 of exemplary disposable operating room drape 10 is joined to first layer of material 11 along at least a portion of second outer periphery 21 from point D1 to point D2 along second outer periphery 21.

In one desired embodiment of the present invention, second layer of material 20 forms cuff 41, which partially covers a lower surface 17 of first layer of material 11, wherein cuff 41 has a closed end 16 and an open end 24 extending across a width of exemplary disposable operating room drape 100 from point D1 to point D2. Open end 24 of cuff 41 may be separated from lower surface 17 of first layer of material 11 to access pocket 25 between second layer of material 20 and first layer of material 11. Cuff 41 and pocket 25 contained therein are desirably sized so as to fit over at least a portion of an operating room table having an opening therein, a table mattress having an opening therein, or both.

Exemplary disposable operating room drape 100 further comprises collar member 800 having an inner periphery 801 (positioned along or near second fenestration 22 in second layer of material 20) and an outer periphery 802 (positioned a substantially equal distance from second fenestration 22 in second layer of material 20), wherein inner periphery 801 of collar member 800 is attached to second layer of material 20 so as to surround second fenestration 22 in second layer of material 20. Collar member 800 may further comprise one or more fasteners 804 positioned along outer periphery 802 and on first surface 805 of collar member 800. Collar member 800 is releasably attachable to first layer of material 11 through first fenestration 13 or both first fenestration 13 and second fenestration 22.

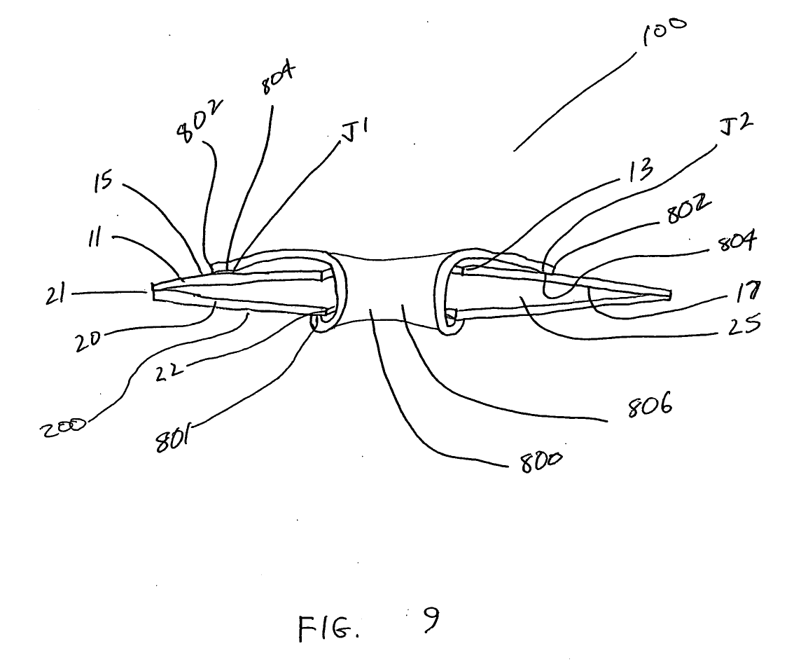

A cross-sectional view of exemplary disposable drape 100 viewed along line G-G of FIG. 6 is provided in FIG. 8. As shown in FIG. 8, exemplary disposable drape 100 comprises collar member 800 attached to second layer of material 20 along second fenestration 22 of second layer of material 20. In FIG. 8, collar member 800 is shown with second periphery 802 of collar member positioned away from second layer of material 20 (i.e., collar member is extending downward from second layer of material 20 due to gravitational forces in this particular figure).

As described above, the disposable operating room drapes of the present invention may comprise a number of components. Further, the above-described exemplary disposable operating room drapes of the present invention may be used in combination with other drapes or other drape components to form draping systems of the present invention. A description of some of the suitable exemplary components is provided below.

I. Disposable Operating Room Draping System Components

The disposable operating room draping systems of the present invention may comprise, but are not limited to, one or more of the following components.

A. Operating Room Drape Components

The disposable operating room draping systems of the present invention comprise an operating room drape such as exemplary disposable operating room drape 10 shown in FIG. 1 or exemplary disposable operating room drape 100 shown in FIG. 6. Exemplary disposable operating room drapes 10 and 100 may comprise one or more of the following components.

1. First Layer of Material

The disposable operating room drapes of the present invention comprises a first layer of material such as exemplary first layer of material 11 of exemplary disposable operating room drape 10 shown in FIGS. 1 and 6. The first layer of material of the disposable operating room drape of the present invention may comprise one or more layers of material bonded to one another. Suitable layers include, but are not limited to, film layers; fabric layers such as woven, nonwoven and knitted fabric layers; foam layers; and combinations thereof.

In one desired embodiment of the present invention, the first layer of material comprises a multi-layer article comprising a lower liquid impervious layer (i.e., the layer that forms lower surface 17 of first layer of material 11), and an upper fiber-containing layer (i.e., the layer that forms upper surface 15 of first layer of material 11) adjacent to the liquid impervious layer. Desirably, the upper fiber-containing layer possesses some degree of liquid absorbency. The liquid impervious layer may comprise a polymeric film-forming material, such as polyethylene, and typically has an average layer thickness of less than about 50 microns (μm) (about 2 mil). Desirably, the polymeric film-forming material comprises a material having a relatively high coefficient of friction or degree of tackiness so that the first layer of material does not slip off of an operating room table mattress during use. One desired polymeric film-forming material having a relatively high coefficient of friction comprises polyethylene.

The fiber-containing layer may comprise a woven or nonwoven fabric layer, or may comprise a layer of fibers adjacent to the liquid impervious layer. The fibers of the fiber-containing layer may comprise a polymeric film-forming material, such as polypropylene, and typically has an average layer thickness of less than about 250 microns (μm) (about 10 mil). In one desired embodiment of the present invention, the fiber-containing layer comprises a polypropylene spunbonded fabric layer having a basis weight of about 1 ounce per square yard.

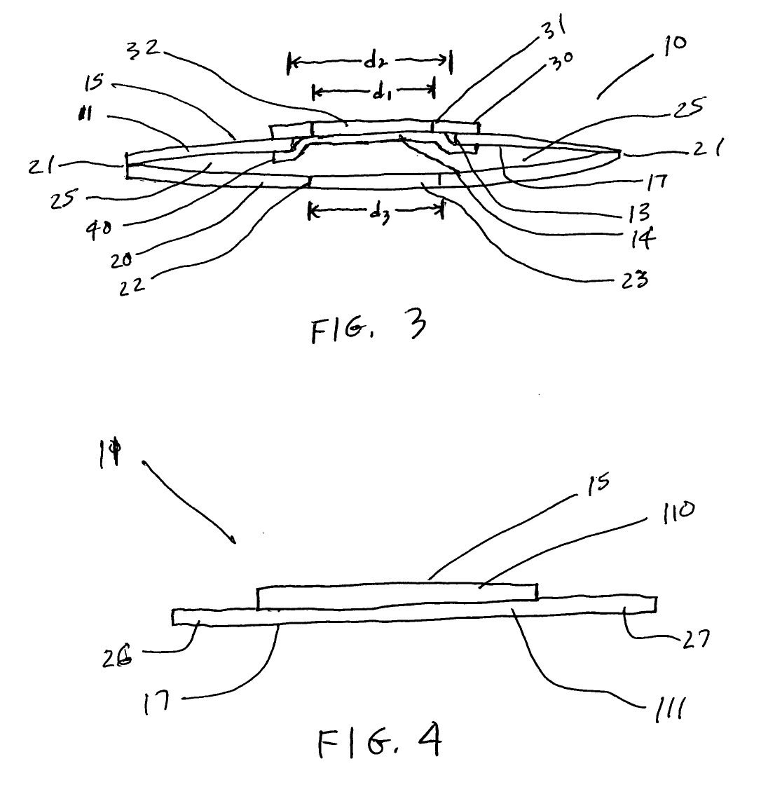

An exemplary two-layered construction suitable for forming first layer of material 11 is shown in FIG. 4. FIG. 4 depicts a cross-sectional view of exemplary first layer of material 11 along line B-B shown in FIG. 1 (or along line H-H shown in FIG. 6). As shown in FIG. 4, exemplary first layer of material 11 comprises upper layer 110 having upper surface 15, and lower layer 111 having lower surface 17. As discussed above, upper layer 110 desirably comprises a fiber-containing layer, such as a nonwoven fabric layer, that possesses some degree of liquid absorbency, and lower layer 111 comprises a liquid impervious layer, such as a polymeric film layer.

Although a two-layered construction as described above may be used in the present invention, in other embodiments, a three or four layered construction may be used wherein at least one layer containing superabsorbent material or particles (SAP) is interposed between the above-described liquid impervious layer and the above-described fiber-containing layer. The SAP-containing layer or layers may further comprise additional components such as fibrous materials (e.g., pulp fibers, synthetic fibers, or a combination thereof), filler materials, or a combination thereof.

The SAP-containing layer or layers may each comprise a substantially uniform distribution of superabsorbent material or particles across an area of the first layer of material, or alternatively, may comprise a non-uniform distribution of superabsorbent material or particles across an area of the first layer of material. For example, a first layer of material useful in the present invention may have a layer of superabsorbent material or particles within a central location of the first layer of material (e.g., an area positioned directly above a central location of an operating room table mattress), but be substantially free of superabsorbent material or particles in an outer periphery of the first layer of material.

Any known superabsorbent material may be used in the first layer of material of the present invention. Suitable commercially available superabsorbent materials include, but are not limited to, superabsorbent materials available from Stockhausen (Greensboro, N.C.) and Dow Chemical (Midland, Mich.).

In a further embodiment of the present invention, the first layer of material of the present invention comprises, in the following order, (i) an outermost fiber-containing layer as described above, (ii) a mesh structure, (iii) a layer containing superabsorbent material or particles, and (iv) a lower layer of liquid impervious material as described above, wherein the mesh structure provides additional tear strength to the first layer of material.

Operating room drapes used in the present invention typically have dimensions so that the operating room table sheet may completely cover an upper surface of an operating room table mattress. In one exemplary embodiment of the present invention, the first layer of material of the operating room drape has a width ranging from about 24 to about 60 inches, and a length ranging from about 48 to about 104 inches. In one desired embodiment, the first layer of material of the operating room drape has a width of about 33 inches, and a length of about 82 inches.

In yet a further embodiment of the present invention, the first layer of material may further comprise an elastic hemline along a portion of the first outer periphery that is not joined to the second layer of material. For example, as shown in FIGS. 2 and 7, first layer of material 11 can have an elastic hemline extending from point F1 along an outer periphery 411 to point F2. The presence of an elastic hemline enables the disposable operating room drape of the present invention to better fit onto an operating table or table mattress similar to the way an elastic hemline in a bedsheet enables the bedsheet to better fit onto a bed mattress.

As shown in FIGS. 1 and 6, first layer of material 11 comprises at least one first fenestration 13 positioned wihtin outer periphery 12. First fenestration 13 has a first fenestration area 14 that may vary in size depending on a number of factors including, but not limited to, the final use of the disposable operating room drape. First fenestration 13 typically has one or more dimensions (i.e., length, width or diameter), wherein each dimension is less than about 24 inches. Typically, first fenestration 13 has one or more dimensions, wherein each dimension ranges from about 6 inches to about 18 inches, and more desirably, ranges from about 8 inches to about 12 inches.

Although not shown in FIGS. 1-2 or 6-7, it should be understood that first layer of material 11 may comprise two or more separate first fenestrations 13 positioned along any portion of upper surface 15 of first layer of material 11. When first layer of material 11 comprises more than one first fenestration 13, each first fenestration 13 may have similar or different dimensions and/or shapes.

2. Adhesive Film

The disposable operating room drape of the present invention also comprises an adhesive film such as exemplary adhesive film 30 of exemplary disposable operating room drape 10 shown in FIG. 1. The adhesive film is adhesively bonded to an upper surface of the first layer of material. The upper surface of the adhesive film typically has very little or no tackiness, while the lower surface (i.e., the surface next to the first layer of material) has an adhesive thereon. A typical construction of the adhesive film comprises an upper backing layer, such as a polymer film, and a lower pressure sensitive adhesive layer on the backing layer. Any polymer film backing layer and any pressure sensitive adhesive may be used in the present invention. In one exemplary embodiment of the present invention, the adhesive film comprises a polyethylene or polyester backing and a pressure sensitive adhesive comprising poly(meth)acrylate; diene rubber such as natural rubber, polyisoprene, and polybutadiene; thermoplastic elastomer; block copolymers such as styrene-isoprene and styrene-isoprene-styrene (SIS) block copolymers, ethylene-propylene-diene polymers, and styrene-butadiene polymers; poly-alpha-olefin; amorphous polyolefin; ethylene-containing copolymer such as ethylene vinyl acetate, ethylacrylate, and ethyl methacrylate; polyurethane; polyamide; epoxy; polyesters; and combinations thereof.

The adhesive film of the disposable operating room drape of the present invention extends over a portion of the first fenestration area of the first fenestration within the first layer of material. As discussed above, at least a portion of the adhesive film is releasably attachable to a second layer of material (described below) through the first fenestration. The adhesive film may overlap a portion of the first fenestration and first fenestration area. In one desired embodiment, the adhesive film completely surrounds the first fenestration and first fenestration area. In this embodiment, the adhesive film further comprises an adhesive film fenestration such that at least a portion of the adhesive film fenestration and a portion of the adhesive film is positioned within an outer periphery of the first fenestration and covering a portion of the first fenestration area.

In one desired embodiment, the adhesive film completely surrounds the first fenestration and first fenestration area and comprises an adhesive film fenestration, wherein an outermost edge of the adhesive film fenestration is positioned within the first fenestration area such that a portion of the adhesive film extends along and completely surrounds an outer edge of the first fenestration area. Such a configuration is shown in FIG. 1. As shown in FIG. 1, adhesive film 30 completely surrounds first fenestration 13. The outermost edge of adhesive film fenestration 31 is positioned completely within first fenestration area 14 such that a portion of adhesive film 30 extends along and completely surrounds an outer edge of first fenestration area 14. Desirably, the outermost edge of adhesive film fenestration 31 (i) is positioned completely within first fenestration area 14, and (ii) extends from about 12.6 mm (0.5 inch) to about 50.8 mm (2.0 inches) into first fenestration area 14 from any edge of first fenestration 13.

The first fenestration and the adhesive film fenestration may have any desired shape including, but not limited to, a circular shape, a rectangular shape, a square shape, a triangular shape, an octagonal shape, etc. Desirably, the first fenestration has a first opening shape, and the adhesive film fenestration has an adhesive film opening shape substantially similar to the first opening shape. For example, the first opening shape and the adhesive film opening shape may each independently be a circular shape. In this embodiment, as discussed above, it is desirable for the first fenestration area ito be greater than an adhesive film fenestration area (i.e., the first fenestration and the adhesive film fenestration have a circular shape, but the adhesive film fenestration circular area is smaller than the first fenestration circular area such that the adhesive film fenestration circular area is positioned completely within the first fenestration area). In one desired embodiment of the present invention, the first fenestration and the adhesive film fenestration have a circular shape, but the adhesive film fenestration has a diameter of about 8 inches, while the first fenestration has a diameter of about 10 inches.

Although not shown in FIGS. 1-2, it should be understood that adhesive film 30 may comprise two or more separate adhesive film fenestrations 31 positioned within adhesive film 30. When adhesive film 30 comprises more than one adhesive film fenestration 31, each adhesive film fenestration 31 may have similar or different dimensions and/or shapes. Desirably, each adhesive film fenestration 31 is aligned with first fenestrations 13 so that outermost edges of each adhesive film fenestration 31 are positioned within first fenestration areas 14 such that portions of adhesive film 30 extend along and completely surround outer edges of each of the first fenestration areas 14.

3. Second Layer of Material

The disposable operating room drape of the present invention may also comprise a second layer of material such as exemplary second layer of material 20 of exemplary disposable operating room drape 10 shown in FIGS. 2 and 7. The second layer of material is desirably joined to the above-described first layer of material along at least a portion of an outer periphery of the second layer of material. For example, as shown in FIGS. 2 and 7, exemplary second layer of material 20 may be joined to first layer of material 11 along any portion of outer periphery 21 and/or along a line extending from point D1 to D2 across lower surface 17 of first layer of material 11.

The second layer of material used in the present invention typically comprises a single-layer material such as a film layer, a paper layer, or a fabric layer. Desirably, the second layer of material used in the present invention comprises a film material, more desirably, a transparent film material. The film material may comprise any polymeric film-forming material, such as polyethylene, and typically has an average layer thickness of less than about 50 microns (μm) (about 2 mil).

The second layer of material further comprises a second fenestration within an outer periphery of the second layer of material. As shown in FIGS. 2 and 7, exemplary second layer of material 20 comprises second fenestration 22 having second fenestration area 23. Desirably, the second fenestration of the second layer of material aligns with or is positioned directly below the first fenestration of the first layer of material, the adhesive film fenestration (when present), or both. Further, the second fenestration of the second layer of material may have any of the above-described opening shapes (e.g., circular, rectangular, etc.). For example, the first fenestration of the first layer of material may have a first opening shape, the second fenestration of the second layer of material may have a second opening shape, and the adhesive film (when present) may have an adhesive film opening shape, wherein each of the first, second, and adhesive film opening shapes is independently a circular shape.

In one desired embodiment of the present invention, the second fenestration is positioned below the adhesive film fenestration and has a second opening shape substantially similar to the adhesive film opening shape. In this embodiment, the second fenestration also desirably has a second fenestration area that is substantially equal to the adhesive film fenestration area, and both the second fenestration area and the adhesive film fenestration area are smaller than the first fenestration area. In such a configuration, an outer protion of the adhesive film (i.e., extending outwardly from beyond an edge of the first fenestration) may be releasaby attached to a corresponding portion of the second layer of material such that a complete seal is made between the adhesive film and the second layer of material. In other words, referring to FIGS. 1-3, the complete seal between the adhesive film and the second layer of material is such that pocket 25 is not accessable from above adhesive film 30 or from below second layer of material 20.

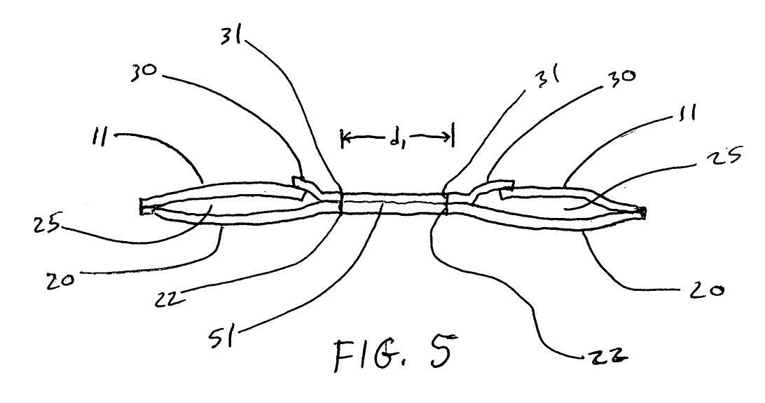

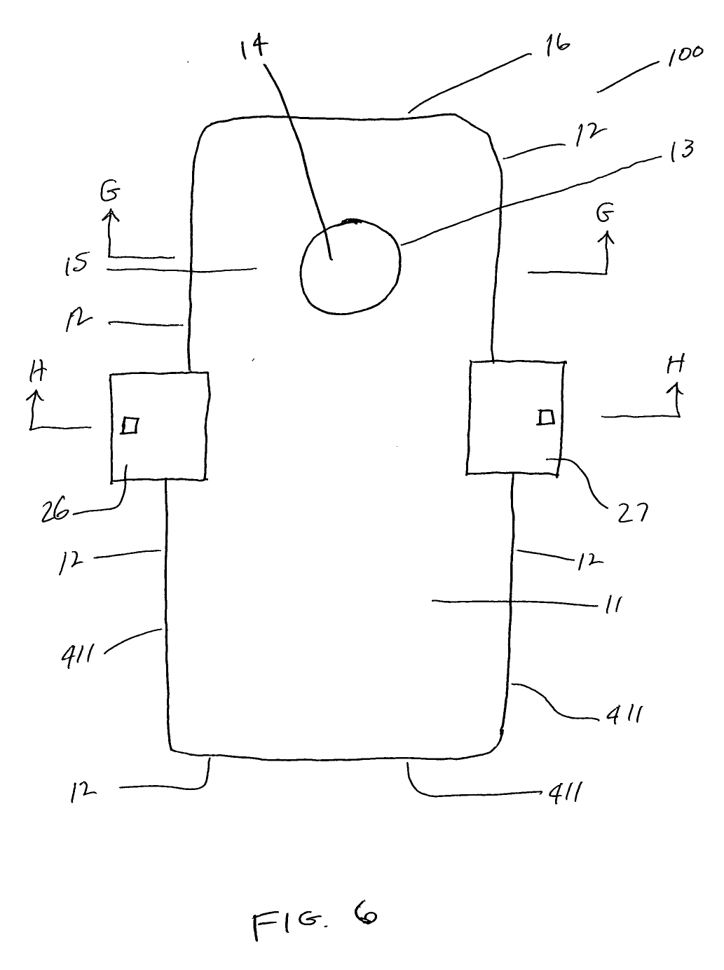

FIG. 5 provides a cross-sectional view of exemplary disposable operating room drape 10 along line A-A shown in FIG. 1 after release liner 40 has been removed from adhesive film 30, wherein adhesive film 30 has been releasably attached to second layer of material 20 so as to form a complete seal 51. As shown in FIG. 5, adhesive film 30 is releasably attached to second layer of material 20 so that edges of adhesive film fenestration 31 and edges of second fenestration 22 are aligned with one another to form complete seal 51. In this exemplary embodiment, each of adhesive film fenestration 31 and second fenestration 22 has a circular opening shape having an opening diameter, d,. Further, as shown in FIG. 5, pocket 25 is not accessable from above adhesive film 30, from below second layer of material 20, or through seal 51.

As discussed above, in one desired embodiment of the present invention, the second layer of material forms a cuff on a lower surface of the first layer of material. The cuff forms a “pocket” on the lower side of the first layer of material. The cuff or pocket is sized so as to fit over at least a portion of an operating room table mattress positioned on an operating room table, or an operating room table mattress in combination with an operating room table. The cuff may be any size such that the cuff fits over an intended substrate.

In one embodiment of the present invention, referring to FIGS. 2 and 7, cuff 41 is formed by joining first layer of material 11 and second layer of material 20 to one another from point D1 along outer periphery 21 of second layer of material 20 to point D2. Cuff 41 has a closed end 16 and an open end 24 extending across a width of exemplary disposable operating room drape 10 from point D1 to point D2. Open end 24 of cuff 41 may be separated from lower surface 17 of first layer of material 11 to access pocket 25 between second layer of material 20 and first layer of material 11. Cuff 41 and pocket 25 contained therein are desirably sized so as to fit over at least a portion of an operating room table having an opening therein, a table mattress having an opening therein, or both.

In one exemplary embodiment, cuff 41 extends across a width of first layer of material 11, and along a length of first layer of material 11 from closed end 16 of cuff 41 at a first end of first layer of material 11 to an open end 24 of cuff 41 at a point between the first end and a second opposite end of first layer of material 11. Typically, cuff 41 and open end 24 of cuff 41 extend across the full width of first layer of material 11. Further, cuff 41 typically extends about ¼ to about ¾ of the length of first layer of material 11.

In one exemplary embodiment, the cuff of the operating room drape has a width (i.e., across the first layer of material) ranging from about 24 to about 60 inches, and a length (i.e., along the length of the first layer of material) ranging from about 18 to about 104 inches. In one desired embodiment, the cuff of the operating room drape has a width of about 33 inches, and a length of about 29 inches.

The cuff feature of the operating room drape enables a single person to quickly and properly cover an operating room table and/or table mattress. In an exemplary method, a person secures the cuff of the drape over the mattress to stabilize the sheet or drape. Then, the person pulls the disposable operating room drape from head to foot to cover the operating room table and/or table mattress. When the operating room drape further comprises an elastic hemline as described above, the operating room drape including a cuff and an elastic hemline fits snugly around an operating room table and/or table mattress.

As with first fenestration 13 described above, second fenestration 22 has a second fenestration area 23 that may vary in size depending on a number of factors including, but not limited to, the final use of the disposable operating room drape. Second fenestration 22 typically has one or more dimensions (i.e., length, width or diameter), wherein each dimension is less than about 24 inches. Typically, second fenestration 22 has one or more dimensions, wherein each dimension ranges from about 4 inches to about 18 inches, and more desirably, ranges from about 6 inches to about 12 inches.

Although not shown in FIGS. 1-2 or 6-7, it should be understood that second layer of material 20 may comprise two or more separate second fenestrations 22 positioned within second layer of material 20. When second layer of material 20 comprises more than one second fenestration 22, each second fenestrations 22 may have similar or different dimensions and/or shapes. Desirably, each second fenestration 22 is aligned with corresponding first fenestrations 13 and adhesive film fenestration 31 (when present) so that outermost edges of each adhesive film fenestration 31 are positioned directly above corresponding outermost edges of each second fenestration 22.

4. Release Liner

The disposable operating room drape of the present invention may further comprise a release liner such as exemplary release liner 40 of exemplary disposable operating room drape 10 shown in FIGS. 2-3. The release liner may be any conventional material having release properties including, but not limited to, a polymeric film having release properties, a polymeric film having a separate release coating thereon, or a paper substrate having a release coating thereon. Further, the release liner may have any dimensions and shape as long as the release liner is able to cover any exposed areas of adhesive film extending over the first fenestration area within the above-described first layer of material.

Suitable release liners that may be used in the present invention include, but are not limited to, silicone coated papers, and silicone coated films, such as polyester films. Examples of suitable commercially available release liners include, but are not limited to, release liners sold under the trade designation AKROSIL™ available from Akrosil Europe (Huerlen, Netherlands) and International Paper (Menasha, Wis.); and release liners available from Daubert Coated Products, Inc. (Dixon, Ill.).

When adhesive film 30 comprises two or more separate adhesive film fenestrations 31, one or more release liners 40 may be used to temporarily cover any exposed areas of adhesive on a lower surface of adhesive film 30.

Although not shown in FIG. 7, one or more of the above-described release liners may be used to temporarily protect one or more fasteners 804 positioned along outer periphery 802 and on first surface 805 of collar member 800 when one or more fasteners 804 comprise areas of pressure sensitive adhesive.

5. Equipment Component Covers of Wings

The disposable operating room drape of the present invention may further comprise one or more equipment component covers or “wings” such as exemplary wings 26 and 27 shown in FIGS. 1-2 and 6-7. Exemplary wings 26 and 27 typically comprise a single-layer material such as a film layer, a paper layer, or a fabric layer. Desirably, each wing used in the present invention comprises a film material, more desirably, a transparent film material. The film material may comprise any polymeric film-forming material, such as polyethylene, and typically has an average layer thickness of less than about 50 microns (μm) (about 2 mil).

Each wing used in the present invention may further comprise a film material having one or more fasteners attached to a surface of the wing. Suitable fasteners include, but are not limited to, hook-and-loop fasteners, and double-sided pressure-sensitive adhesive tapes. Such fasteners may be used to releasably attach a portion of a wing to other surfaces including, but not limited to, a surface of an operating room drape, a surface of an operating room table or mattress, a surface of any other piece of equipment in an operating room, or a combination thereof. Exemplary fasteners 28 and 29 are shown in FIGS. 2 and 7 on a lower surface of wings 26 and 27 respectively. In this exemplary embodiment, fasteners 28 and 29 comprise double-sided pressure-sensitive adhesive tapes.

Wings used in the present invention typically have dimensions so that the wing substantially covers an area extending downward and/or outward from the operating room drape. In one exemplary embodiment of the present invention, each wing has a width (i.e., the dimension extending along a length of the operating room drape) of up to about 48 inches, and a length (i.e., the dimension extending outward from the operating room drape) of up to about 48 inches. In one desired embodiment, each wing has a width of about 16 inches, and a length of about 9 inches.

Typically, a given disposable operating room drape of the present invention comprises two wings on either side of an operating room drape. In one desired embodiment of the present invention, the disposable operating room drape of the present invention comprises two substantially identical transparent wings on either side of the operating room drape, wherein the wings are simply an extension of a lower layer used to form the first layer of material. For example, wings 26 and 27 shown in FIGS. 1 and 6 may comprise a liquid impervious polymeric film material, such as lower layer 111, as shown in FIG. 4.

6. Collar Members In one exemplary embodiment of the present invention, the disposable operating room drape further comprises at least one collar member such as exemplary collar member 800 shown in FIG. 7. Exemplary collar member 800 typically comprises a single-layer material such as a film layer, a paper layer, or a fabric layer. Desirably, each collar member used in the present invention comprises a film material, more desirably, a transparent or semi-transparent film material. The film material may comprise any polymeric film-forming material, such as polyethylene or ethylene copolymer, and typically has an average layer thickness of less than about 50 microns (μm) (about 2 mil).

In one desired embodiment of the present invention, the collar member comprises a polymer film that is capable of being stretched at least 5% without breaking. Desirably, the stretchable polymer film comprises an ethylene methyl acrylate copolymer.

Typically, the collar member is attached to the second layer of material of the disposable drape so that the collar member surrounds the second fenestration of the second layer of material. The collar member may be positioned along, near or adjacent to the second fenestration of the second layer of material on either outer surface of the second layer of material. Desirably, the collar member is positioned along, near or adjacent to the second fenestration on an outer surface of the second layer opposite the first layer of material so that the collar member extends through the second fenestration and the first fenestration when the collar member is releasably attached to the first layer of material.

Each collar member may have a collar length (i.e., the distance from an inner periphery to an outer periphery of the collar member) as desired for a given application. Typically, each collar member has a collar length ranging from about 5.1 cm (2.0 in) to about 30.5 cm (12.0).

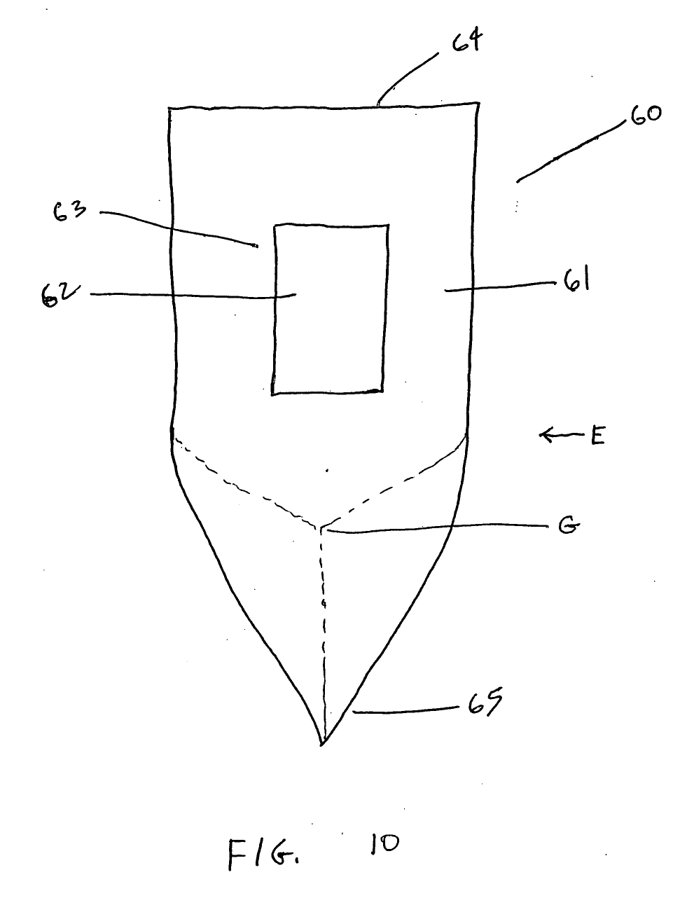

As shown in FIG. 7, each collar member used in the present invention may comprise a film material having one or more fasteners attached to a surface of the collar member. Suitable fasteners include, but are not limited to, hook-and-loop fasteners, and double-sided pressure-sensitive adhesive tapes. Desirably, the one or more fasteners comprise areas of double-sided pressure-sensitive adhesive tape. Such fasteners may be used to releasably attach a portion of the collar member to other surfaces including, but not limited to, a surface of the first layer of material as described above and as shown in FIG. 9.

As shown in FIG. 9, exemplary collar member 800 is attached to second layer of material 20 of disposable drape 100 along an outer surface 200 of second layer of material 20 surrounding second fenestration 22. Collar member 800 is attached to second layer of material 20 and extends through second fenestration 22 and first fenestration 13 of first layer of material 11. Outer periphery 802 of collar member 800 is attached to outer surface 15 of first layer of material 11 via fasteners 804 at points J1 and J2 as shown in FIG. 9. In this configuration, a sterile field is formed in an area surrounded by surface 806 of collar member 800.

B. Secondary Drape Component

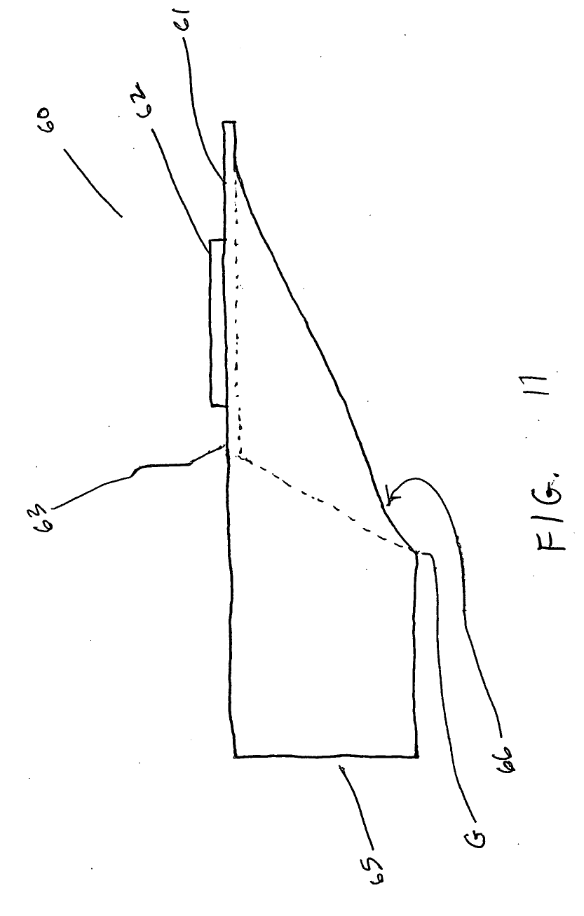

The above-described disposable operating room drape may be alone or in combination with at least one additional drape such as exemplary drape 60 shown in FIG. 10. The drape combination forms a draping system that can (i) further provide a variety of sterile fields within an operating room setting, (ii) further provide barrier protection (i.e., a barrier from liquids, germs, etc.) for a patient and/or piece of equipment in an operating room, or (iii) both (i) and (ii).

Typically, exemplary drape 60 used in the present invention comprise a multi-layer material comprising a liquid impervious layer 61, and a fiber-containing layer 62 adjacent to and on an upper surface 63 of liquid impervious layer 61. Typically, liquid impervious layer 61 and a fiber-containing layer 62 are formed from materials similar to the materials used to form the above-described first layer of material. The liquid impervious layer may comprise a polymeric film-forming material, such as polyethylene, and typically has an average layer thickness of less than about 50 microns (μm) (about 2 mil). The fiber-containing layer may comprise a woven or nonwoven fabric layer, or may comprise a layer of fibers. The fibers of the fiber-containing layer may comprise a polymeric film-forming material, such as polypropylene, and typically has an average layer thickness of less than about 250 microns (μm) (about 10 mil). Desirably, fiber-containing layer 62 comprises a polypropylene spunbonded fabric layer having a basis weight of about 1 ounce per square yard.

In one desired embodiment of the present invention, the at least one additional drape, such as exemplary drape 60, comprises a first end 64 having a flat structure, and an opposite collar end 65 formed from the above-described multi-layer material, wherein liquid impervious layer 61 forms an inner surface of a collar cavity 66 at collar end 65. In this embodiment, exemplary drape 60 may be formed by slitting a collar structure comprising liquid impervious layer 61 with or without fiber-containing layer 62 from a first end 64 of the collar structure to a point G along the collar structure. Fiber-containing layer 62 may then be bonded to liquid impervious layer 61 via a thermal bonding process and/or via an adhesive. In this embodiment, the outer circumference of collar end 65 of exemplary drape 60 is substantially equal in length to a width of exemplary drape 60 at first end 64.

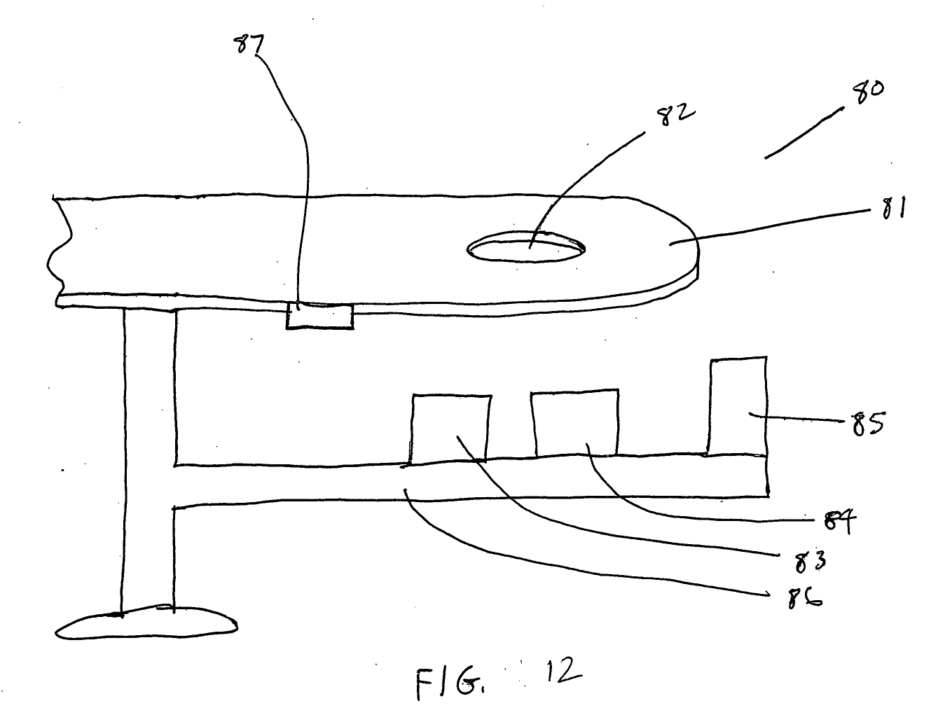

FIG. 11 provides a side view of exemplary drape 60 from point E shown in FIG. 10. As shown in FIG. 11, collar cavity 66 at collar end 65 of exemplary drape 60 is accessible by separating outer edges of liquid impervious layer 61 between point G and first end 64.

The at least one additional drape, such as exemplary drape 60, may be opened (i.e., collar cavity 66 is expanded), and then positioned over and around an object, such as a piece of equipment near and/or under an operating room table. The at least one additional drape is sized so as to fit over one or more pieces of equipment in an operating room. In one embodiment of the present invention, the at least one additional drape has a width (i.e., a full width as measured at first end 64) of up to 96 inches, and a length of up to 120 inches. Typically, the at least one additional drape has a width ranging from about 36 inches to about 72 inches, and a length ranging from about 60 inches to about 96 inches.

The at least one additional drape, such as exemplary drape 60, is particularly useful for covering operating room equipment in an operating room, such that fiber-containing layer 62 is positioned directly under an area wherein a surgical procedure is being performed. In this embodiment, any fluids (i.e., body fluids, blood, etc.) may fall directly on fiber-containing layer 62 and be absorbed into fiber-containing layer 62. Typically, fiber-containing layer 62 of the at least one additional drape, such as exemplary drape 60, occupies less than the full surface area of liquid impervious layer 61 as shown in FIG. 10. For example, when the at least one additional drape (and liquid impervious layer 61) has a width ranging from about 36 inches to about 72 inches, and a length ranging from about 60 inches to about 96 inches, fiber-containing layer 62 will typically have a width ranging from about 8 inches to about 24 inches, and a length ranging from about 12 inches to about 36 inches. In one desired embodiment of the present invention, the at least one additional drape hacomprises (i) liquid impervious layer 61 having a width about 60 inches, and a length of about 72 inches, and (ii) fiber-containing layer 62 thereon having a width about 12 inches, and a length of about 24 inches.

Fiber-containing layer 62 may be positioned along any portion of upper surface 63 of exemplary drape 60. In one desired embodiment, fiber-containing layer 62 is positioned along a portion of upper surface 63 at first end 64 of exemplary drape 60, opposite collar end 65. Although not shown in FIGS. 10-11, it should be understood that two or more separate fiber-containing layers 62 may be positioned along upper surface 63 of exemplary drape 60.

C. Additive Draping System Components

Any of the above-described disposable operating room draping systems of the present invention may further comprise one or more additional components including, but not limited to, disposable bags with or without bag closure devices, additional equipment covers, and combinations thereof. Exemplary equipment covers include, but are not limited to, transparent covers having a desired shape (e.g., a circular shape) and an elastic seam extending along at least a portion of the periphery of the cover.

D. Component Additives

Any of the above-described disposable operating room draping system components of the present invention may further comprise one or more additives coated onto or incorporated in one or more of the materials used to form the individual component. Suitable additives include, but are not limited to, antimicrobial agents, colorants, additives to increase the coefficient of friction of a given component layer, etc. In one desired embodiment of the present invention, one or more components of the disposable operating room surgical draping system comprises an antimicrobial agent incorporated therein. Suitable antimicrobial agents include, but are not limited to, triclosan and other antimicrobial agents commercially available under the trade designation MICROBAN®.

For example, one or more of adhesive film 30, first layer of material 11, second layer of material 20, liquid impervious layer 61, and fiber-containing layer 62 may contain one or more of the above-mentioned additives, such as antimicrobial agents commercially available under the trade designation MICROBAN®.

II. Methods of Making Disposable Operating Room Draping Systems

The present invention is further directed to methods of making an operating room drape and operating room draping systems. Any of the above-described individual components used to form the operating room drapes and operating room draping systems of the present invention may be formed using conventional methods. For example, liquid impervious film layers may be forming via any film-forming process including, but not limited to, a film extrusion process, a film-blowing process, etc. Fiber-containing layers, such as a woven fabric layer or a nonwoven fabric layer, may be formed using conventional weaving or web-forming processes including, but not limited to, meltblowing processes, spunbonding processes, spunlacing processes, hydroentangling processes, etc.

Films and fabric layers may be joined to one another along outer peripheries of each web using any conventional bonding technique including, but not limited to, thermal bonding processes, adhesive bonding, etc. In one exemplary embodiment of the present invention, a liquid impervious layer may be bonded to a nonwoven fabric layer using a conventional point-bonding apparatus, wherein thermal bonds are used to join the liquid impervious layer to the nonwoven fabric layer. The degree of bonding, size of individual point bonds, and concentration of point bonds may vary as desired.

In exemplary disposable drapes comprising a collar member, the collar member may be bonded to the second layer of material using any conventional bonding technique including such as those described above. In on desired embodiment, the collar member is bonded to the second layer of material using a thermal bonding process or a heat-activatable adhesive.

Fenestrations may be cut into any of the above-mentioned substrates using conventional cutting processes including, but not limited to, a stamping process, etc.

III. Methods of Using Disposable Operating Room Draping Systems

The present invention is further directed to methods of preparing an operating room substrate for a surgical procedure. In one exemplary embodiment of the present invention, the method of preparing an operating room substrate comprises the steps of placing a disposable drape of the present invention onto an outer surface of a substrate having a substrate fenestration therein; aligning a first fenestration of the disposable drape with the substrate fenestration of the operating room substrate; and releasably attaching a portion of a collar member extending from a second layer of material of the disposable drape beneath the operating room substrate to a first layer of material of the disposable drape through the first fenestration and the substrate fenestration. The operating room substrate may comprise an operating room table, a vertically extending substrate, or any other substrate having a substrate fenestration therein.

In another exemplary embodiment of the present invention, the method of preparing an operating room substrate comprises the steps of placing a disposable drape of the present invention onto an outer surface of a substrate having a substrate fenestration therein; aligning a first fenestration of the disposable drape with the substrate fenestration of the operating room substrate; and releasably attaching a portion of an adhesive film located on a first layer of material of the disposable drape to a second layer of material of the disposable drape through the first fenestration and the substrate fenestration.

The present invention is even further directed to methods of creating a sterile field in an operating room setting. The sterile field may be near or on a patient, near or on a piece of equipment, or through a piece of equipment, such as an operating room table. In one exemplary embodiment of the present invention, the method of creating a sterile field comprises the steps of placing a disposable drape of the present invention onto an outer surface of an operating room substrate having a substrate fenestration therein; aligning a first fenestration of the disposable drape with the substrate fenestration of the operating room substrate; releasably attaching a portion of a collar member located on a second layer of material of the disposable drape (on one side of the operating room substrate) to a first layer of material of the disposable drape (on an opposite side of the operating room substrate) through the first fenestration and the substrate fenestration; and extending a portion of a patient's body through the first fenestration and the substrate fenestration. In one exemplary embodiment of the present invention, the operating room substrate comprises an operating room table.

In a further exemplary embodiment of the present invention, the method of creating a sterile field comprises the steps of placing a disposable drape of the present invention onto an outer surface of an operating room substrate having a substrate fenestration therein; aligning a first fenestration of the disposable drape with the substrate fenestration of the operating room substrate; releasably attaching a portion of an adhesive film located on a first layer of material of the disposable drape to a second layer of material of the disposable drape through the first fenestration and the substrate fenestration; and extending a portion of a patient's body through the first fenestration and the substrate fenestration. In this exemplary embodiment of the present invention, the operating room substrate may also comprise an operating room table.

In one desired embodiment of the present invention, the disposable operating room draping system of the present invention is used to create a sterile field through an operating table as shown in FIGS. 12-14. As shown in FIG. 12, exemplary operating room apparatus 80 comprising operating room table 81 having table fenestration 82 therein. Exemplary operating room apparatus 80 further comprises equipment pieces 83, 84 and 85 along support member 86, and controls 87 positioned along both sides of operating room table 81. Such an operating room apparatus 80 may be used to perform surgical procedures including, but not limited to, breast brachytherapy procedures, breast biopsy scanning procedures, surgical procedures on a patient's appendage, eye, etc. Commercially available operating room apparatus similar to exemplary operating room apparatus 80 include, but are not limited to, the MammoTest™ Select™ Breast Biopsy System commercially available from Fischer Imaging Corporation (Denver, Colo.).

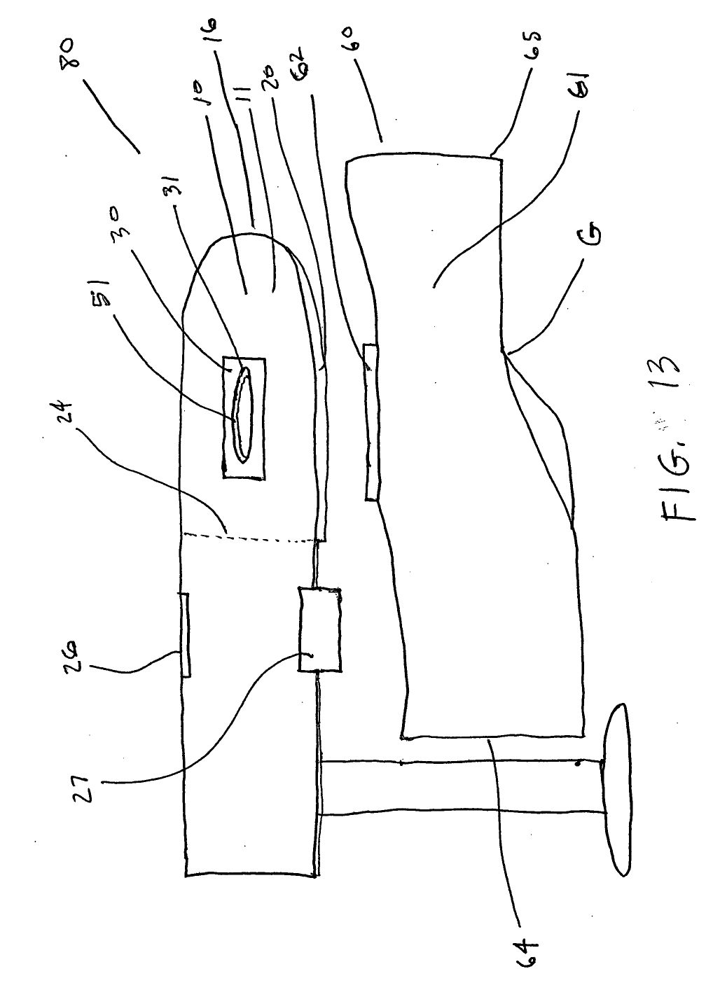

FIG. 13 depicts a side view of exemplary operating room apparatus 80 wherein portions of exemplary operating room apparatus 80 are covered by an exemplary draping system of the present invention. As shown in FIG. 13, operating room table 81 is covered with exemplary disposable operating room drape 10 of FIG. 1, while equipment pieces 83, 84 and 85, and support member 86 are covered with exemplary additional drape 60 of FIG. 10. As shown in FIG. 13, exemplary disposable operating room drape 10 is draped over operating room table 81 such that adhesive fenestration 31 is aligned with and over table fenestration 82. Wings 26 and 27 cover controls 87 on opposite sides of operating room table 81. Second layer of material 20 can be seen along first periphery 12 of first layer of material 11. A dashed line running across operating room table 81 is used to designate where open end 24 of cuff 41 is located. As shown in FIG. 13, cuff 41 fits over the portion of operating room table 81 extending from open end 24 to first end 16 of exemplary disposable operating room drape 10. Further, complete seal 51 can be seen within adhesive film fenestration 31. As discussed above, complete seal 51 is formed by releasably attaching the exposed adhesive surface of adhesive film 30 to second layer of material 20 so that edges of adhesive film fenestration 31 and edges of second fenestration 22 within second layer of material 20 are aligned with one another.

Further, as shown in FIG. 13, fiber-containing layer 62 of exemplary additional drape 60 is positioned directly below adhesive film fenestration 31 and table fenestration 82. In this position, fiber-containing layer 62 may be used to absorb any fluids falling from an area directly below adhesive film fenestration 31 and table fenestration 82. As discussed above, desirably liquid impervious layer 61 of exemplary additional drape 60 and wings 26 and 27 are each formed of a transparent polymeric film material so that equipment pieces 83, 84 and 85, and controls 87 can be seen through liquid impervious layer 61 and wings 26 and 27.

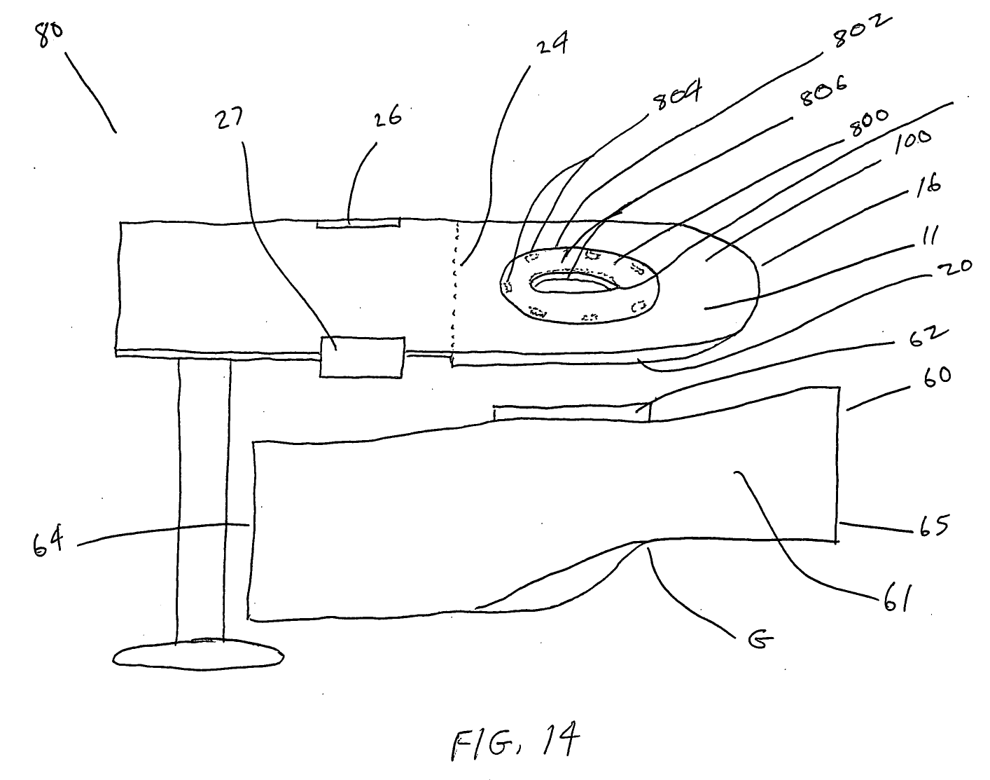

In a further embodiment shown in FIG. 14, portions of exemplary operating room apparatus 80 are covered by an exemplary draping system of the present invention. As shown in FIG. 14, operating room table 81 is covered with exemplary disposable operating room drape 100 of FIG. 6, while equipment pieces 83, 84 and 85, and support member 86 are covered with exemplary additional drape 60 of FIG. 10. As shown in FIG. 14, exemplary disposable operating room drape 100 is draped over operating room table 81 such that first fenestration 13 (covered by collar member 800) is aligned with and over table fenestration 82. Wings 26 and 27 cover controls 87 on opposite sides of operating room table 81. Second layer of material 20 can be seen along first periphery 12 of first layer of material 11. A dashed line running across operating room table 81 is used to designate where open end 24 of cuff 41 is located. As shown in FIG. 14, cuff 41 fits over the portion of operating room table 81 extending from open end 24 to first end 16 of exemplary disposable operating room drape 10.

As shown in FIG. 14, exemplary disposable operating room drape 100 is capable of forming a sterile field within and through table fenestration 82 due to collar member 800. Collar member 800 extends from second layer of material 20 (below operating room table 81), through table fenestration 82 and first fenestration 13 (covered by collar member 800), and is attached to an upper surface of first layer of material 11 to form the sterile field. One or more fasteners 804 are shown as dashed lines along outer periphery 802 of collar member 800.

Further, as shown in FIG. 14, fiber-containing layer 62 of exemplary additional drape 60 is positioned directly below first fenestration 13 (covered by collar member 800) and table fenestration 82. In this position, fiber-containing layer 62 may be used to absorb any fluids falling from an area directly below first fenestration 13 (covered by collar member 800) and table fenestration 82. As discussed above, desirably liquid impervious layer 61 of exemplary additional drape 60 and wings 26 and 27 are each formed of a transparent polymeric film material so that equipment pieces 83, 84 and 85, and controls 87 can be seen through liquid impervious layer 61 and wings 26 and 27.

The present invention is further directed to methods of preparing an operating room table using any one of the above-described disposable operating room drapes or draping systems. In one exemplary embodiment of the present invention, the method comprises the steps of placing a disposable operating room drape onto an upper surface of an operating room table having a table fenestration therein; fitting a first end portion of the disposable operating room drape over a first end of the operating room table or a mattress thereon; aligning a first fenestration within the first layer of material of the disposable operating room drape with the table fenestration; and unfolding the disposable operating room drape to cover a remaining portion of the operating room table or a mattress thereon. The exemplary method may further comprises one or more of the following steps:

(1) aligning a second fenestration within the second layer of material of the disposable operating room drape with the table fenestration;

(2) partially enclosing a portion of a first end of the operating room table or a mattress thereon with a cuff of the disposable operating room drape when the cuff is present;

(3) fitting an opposite end portion (i.e., opposite the first end portion) of the disposable operating room drape over an opposite end of the operating room table or a mattress thereon using an elastic hemline surronding a portion of an outer periphery of the disposable operating room drape;

(4) attaching wing portions of the disposable operating room drape to the operating room table or other equipment and/or controls adjacent to the operating room table;

(5) removing one or more release liners from a lower surface of the adhesive film (when present);

(6) releasably attaching a portion of the adhesive film located on a first layer of material of the disposable drape to a second layer of material of the disposable drape through the first fenestration and the table fenestration;

(7) removing one or more release liners from outer surface(s) of one or more areas of pressure sensitive adhesive along an outer periphery of a collar member (when present); and

(8) releasably attaching a portion of the collar member located on a second layer of material of the disposable drape to a first layer of material of the disposable drape through (i) the first fenestration of the first layer of material and (ii) the table fenestration.

In addition to the above-described disposable operating room drapes and draping systems, the present invention is directed to an operating room table in combination with any of the above-described disposable operating room drapes and draping systems. Further, the present invention is directed to a packaged disposable operating room surgical draping system comprising any of the above-described disposable operating room drapes and draping systems, and a kit lining or packaging material enclosing the disposable operating room drapes and draping systems.

The present invention is described above and further illustrated below by way of claims, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A disposable drape comprising:
a first layer of material having a first outer periphery and a first fenestration having a first fenestration area positioned within the first outer periphery;
a second layer of material having a second outer periphery and a second fenestration positioned within the second outer periphery, wherein the second layer of material is joined to the first layer of material along a portion of the second outer periphery; and
a collar member having an inner periphery and an outer periphery, wherein the inner periphery of the collar member is attached to the second layer of material so as to surround the second fenestration of the second layer of material;
wherein said collar member is releasably attachable to the first layer of material.

2. The disposable drape of claim 1, further comprising one or more fasteners attached along the outer periphery of the collar member.

3. The disposable drape of claim 2, wherein the one or more fasteners comprise one or more areas of pressure sensitive adhesive.

4. The disposable drape of claim 1, wherein the collar member extends through the first and second fenerstrations, and is attached to an upper surface of the first layer of material.

5. The disposable drape of claim 1, wherein the first layer of material comprises an uppermost layer of nonwoven fabric material, and a lower layer of liquid impermeable film.

6. The disposable drape of claim 5, wherein the first layer of material further comprises wings extending outward from opposite sides of the first layer of material, said wings comprising portions of the lower layer of liquid impermeable film.

7. The disposable drape of claim 1, wherein said collar member comprises a polymer film.

8. The disposable drape of claim 7, wherein the polymer film is capable of being stretched at least 5% without breaking.

9. The disposable drape of claim 7, wherein the polymer film comprises ethylene methyl acrylate copolymer.

10. The disposable drape of claim 1, wherein said collar member has a collar length ranging from about 5.1 cm (2.0 in) to about 30.5 cm (12.0).

11. The disposable drape of claim 1, in combination with a second disposable drape, wherein the second disposable drape comprises:
a third layer of material having a third outer periphery; and
a fourth layer of material attached to the third layer of material and positioned within the third outer periphery, said fourth layer of material being capable of absorbing liquid.

12. A method of covering a substrate having a substrtae fenestration therein, said method comprising the steps of:
placing the disposable drape of claim 1 onto an outer surface of the substrate having a substrate fenestration therein;
alligning the first fenestration with the substrate fenestration; and
releasably attaching the collar member to the first layer of material through the first fenestration and the substrate fenestration.

13. A disposable drape comprising:
a first layer of material having a first outer periphery and a first fenestration having a first fenestration area positioned within the first outer periphery;
a second layer of material having a second outer periphery and a second fenestration positioned within the second outer periphery, wherein the second layer of material is joined to the first layer of material along a portion of the second outer periphery; and
an adhesive film attached to an upper surface of the first layer of material and extending over a portion of the first fenestration area, wherein said adhesive film is releasably attachable to the second layer of material through the first fenestration.

14. The disposable drape of claim 13, further comprising a release liner applied over any portion of the adhesive film extending over the first fenestration area, said release liner being positioned between a lower surface of the first layer of material and an upper surface of the second layer of material.

15. The disposable drape of claim 13, wherein said adhesive film completely surrounds the first fenestration and further comprises an adhesive film fenestration positioned within an outer periphery of the first fenestration.

16. The disposable drape of claim 15, wherein an outermost edge of the adhesive film fenestration is positioned within the first fenestration area such that a portion of the adhesive film extends along and completely surrounds an outer edge of the first fenestration area.

17. The disposable drape of claim 16, wherein the first fenestration has a first opening shape, and the adhesive film fenestration has an adhesive film opening shape substantially similar to the first opening shape, and wherein the first fenestration area is greater than an adhesive film fenestration area.

18. The disposable drape of claim 17, wherein the first opening shape and the adhesive film opening shape each independently have a circular shape.

19. The disposable drape of claim 17, wherein the second fenestration is positioned below the adhesive film fenestration and has a second opening shape substantially similar to the adhesive film opening shape, said second fenestration having a second fenestration area substantially equal to the adhesive film fenestration area.

20. The disposable drape of claim 19, wherein the first opening shape, the second opening shape and the adhesive film opening shape each independently have a circular shape.

21. The disposable drape of claim 13, wherein said adhesive film is releasably attached to the second layer of material through the first fenestration.

22. The disposable drape of claim 13, wherein the first layer of material comprises an uppermost layer of nonwoven fabric material, and a lower layer of liquid impermeable film.

23. A method of covering a substrate having a substrtae fenestration therein, said method comprising the steps of:
placing the disposable drape of claim 13 onto an outer surface of the substrate having a substrate fenestration therein;
alligning the first fenestration with the substrate fenestration; and
releasably attaching a portion of the adhesive film to the second layer of material through the first fenestration and the substrate fenestration.

24. A disposable drape comprising:
an operating room drape having a cuff at one end and on a lower surface of the operating room drape, said cuff being sized so as to be capable of partially enclosing a portion of an operating room table mattress, wherein the operating room drape has a first fenestration therein, and the cuff has a second fenestration therein, said first and second fenestrations being aligned with one another.
